(19) United States
(12) Patent Application Publication
(10) Pub. No.: US 2012/0271117 A1
(43) Pub. Date: Oct. 25, 2012

(54) INCISION FIXATION DEVICE FOR SINGLE SITE LAPAROSCOPY

(76) Inventor: Chin-Cheng HSIEH, New Taipei City (TW)

(21) Appl. No.: 13/091,668

(22) Filed: Apr. 21, 2011

(51) Int. Cl. A61B 1/32

(52) U.S. Cl. 600/208

(57) ABSTRACT

An incision fixation device for single site laparoscopy is coupled with a sleeve to be positioned on a surgical incision. The sleeve has one isolation portion containing a passage. The fixation device includes a bracing member located in the passage, a compact member coupled to the isolation portion, and an upper lid containing a plurality of guiding portions and a second engaging portion. The bracing member has an anchor end and a first engaging portion. The compact member has at least one holding portion to hold the anchor end with the isolation portion interposed therebetween. The second engaging portion is releasably engaged with the first engaging portion. Each guiding portion has an instrument passage to guide a surgical instrument to pass through the surgical incision. The compact member has a first and a second fastening mechanisms that are movable against each other to press the anchor end and isolation portion.

### FIELD OF THE INVENTION

The present invention relates to an incision fixation device for single site laparoscopy and particularly to a device to guide a surgical instrument to pass through a surgical incision.

### BACKGROUND OF THE INVENTION

In the past, a surgical operation was constrained by technology and equipment, and a large incision has to be made on a patient's body to perform the operation. Because of the large incision, a greater amount of patient's blood could be lost, more portions of patient's body are affected, and much time is needed to cure the wound.

With constant progress of technology and equipment, minimally invasive surgery has been gradually developed and widely applied to extensive fields in recent years. Equipment for such surgery also has a great deal of progress and improvements. For instance, R.O.C. publication No. 201041557 entitled “Multi-port mask apparatus for a single site laparoscope” discloses a mask apparatus for a laparoscopic surgical operation that includes a body and a head. The body includes a hollow sleeve which has a lower anchor portion, an upper anchor portion, and a pulling strip at the bottom of the hollow sleeve. The head has a gripping portion coupled on the outer side of the upper anchor portion and a holding portion connected to the gripping portion. The holding portion has a plurality of coupling ports. As shown in its drawings, before the mask apparatus is used in surgery, an incision has to be made on a patient's body; then the lower anchor portion of the sleeve is inserted into the incision with at least a portion of the hollow sleeve wedged inside the patient's body; by pulling the pulling strip, the lower anchor portion can be driven to butt the inner side of patient's abdomen. The upper anchor portion and most of the hollow sleeve are exposed outside the outer skin of patient's abdomen. To securely hold the upper anchor portion above the incision and prevent the rest of the hollow sleeve from hindering surgical operation, the hollow sleeve must be rolled upwards manually from the upper anchor portion as shown in its FIG. 7. Finally, the head (gripping portion) is coupled to the upper anchor portion and the rolled hollow sleeve to position the head on the incision. Then various kinds of surgical instruments can be inserted into the abdominal cavity through the coupling ports to perform the surgical operation.

While the aforesaid prior art can brace the incision by positioning the hollow sleeve thereon to facilitate surgical operation, it still has drawbacks, notably:

1. The extra length of the hollow sleeve has to be rolled up manually. It burdens the surgical operation in the operation room with insufficient human resources. Moreover, rolling the hollow sleeve creates unnecessary tearing on the incision that is unfavorable for curing after the surgery.

2. After the hollow sleeve is positioned, the head is pushed to allow the gripping portion to latch on the upper anchor portion. But this also creates troublesome work for the medical personnel and unnecessary tearing on the incision as the head mounted onto the upper anchor portion.

3. During the minimally invasive surgery, parts of body tissues might be resected and removed. However, the coupling ports of the head hinder the removal action, hence the head and upper anchor portion must be separated temporarily to remove the resected body tissues. As mentioned above, disassembly and assembly of the head cannot be easily done. During surgical operation, any unnecessary actions should be avoided. The aforesaid conventional technique creates such trouble for medical personnel involved.

4. The lower anchor portion is connected to a pulling strip which has a certain width that could also tear the incision during pulling and create unnecessary injury to the incision.

All these show that there is still room for improvement.

### SUMMARY OF THE INVENTION

In view of medical personnel are insufficiently staffed and have to bear heavy workload during surgery, and unnecessary actions have to be avoided to hurt the patient, and the conventional mask apparatus still leaves a lot to be desired when in use, such as inconvenient installation, incision tearing during the installation, and difficult removal of the resected tissues, the primary object of the present invention is to provide a surgical instrument that can be easily installed, and assembled or disassembled quickly.

The present invention provides an incision fixation device for single site laparoscopy coupled with a sleeve to be positioned on a surgical incision. The sleeve has at least one isolation portion which forms a passage. The fixation device includes a bracing member held in the passage, a compact member coupled to an outer side of the isolation portion, and an upper lid containing a plurality of guiding portions and a second engaging portion. The bracing member has an anchor end and a first engaging portion. The compact member has at least one holding portion on the inner rim to hold the anchor end with the isolation portion interposed therebetween. The second engaging portion is releasably engaged with the first engaging portion. Each guiding portion has an instrument passage communicating with the passage to guide a surgical instrument to pass through the surgical incision to perform the surgical operation. The compact member has two ends equipped respectively with a first fastening mechanism and a second fastening mechanism that are movable against each other to drive the compact member to contract inwards and press the anchor end and the isolation portion through the holding portion.

The fixation device thus formed can create tight coupling between the isolation portion and the anchor end or loosening thereof by fastening or loosening the compact member, thereby to determine the relative positions of the bracing member and upper lid against the isolation portion. Moreover, the upper lid and bracing member can be assembled or disassembled quickly. When removing the resected body tissues is required during a surgery, the upper lid can be disassembled to meet such purpose. The actions of assembly and disassembly do not apply force upon the incision, thus do not harm the incision. By means of aforesaid structure of the invention, procedures of installing the surgical instrument on the incision can be simplified, and the burden of medical personnel also can be reduced. In addition, removing the resected body tissues during surgery or assembly or disassembly of the fixation device does not produce significant tearing or pressure on the incision, thus can help patient to recover more quickly.

The foregoing, as well as additional objects, features and advantages of the invention will be more readily apparent from the following detailed description, which proceeds with reference to the accompanying drawings.

### BRIEF DESCRIPTION OF THE DRAWINGS

FIG. 1 is an exploded view of the incision fixation device of the invention.

FIG. 2 is an exploded view of the upper lid and bracing member.

FIG. 3 is a schematic view of the incision fixation device and the sleeve in a coupled condition.

FIG. 4 is a sectional view of the incision fixation device and the sleeve in a coupled condition.

FIG. 5A is a schematic view of the first and second fastening mechanisms in a use condition.

FIG. 5B is a schematic view of the first and second fastening mechanisms in another use condition.

### DETAILED DESCRIPTION OF THE PREFERRED EMBODIMENTS

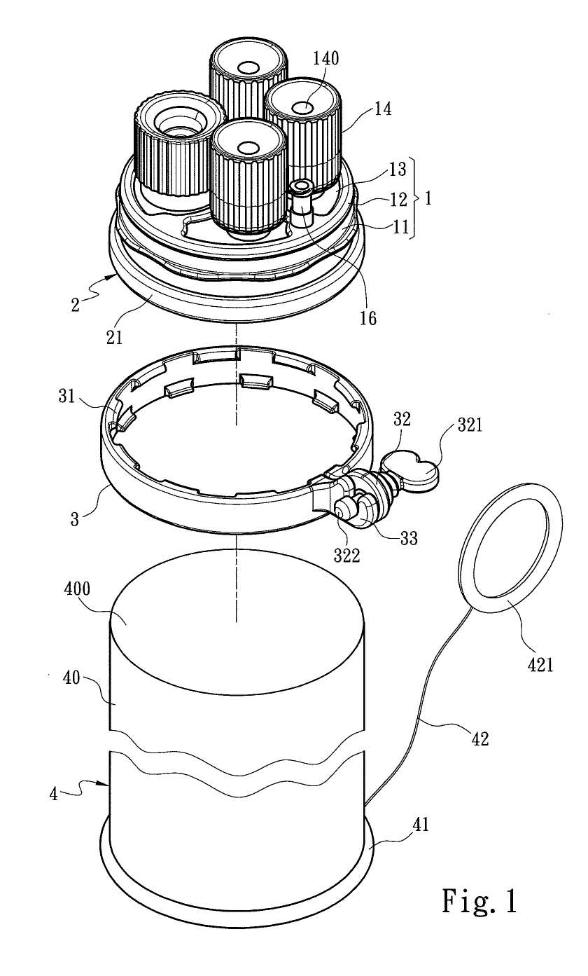

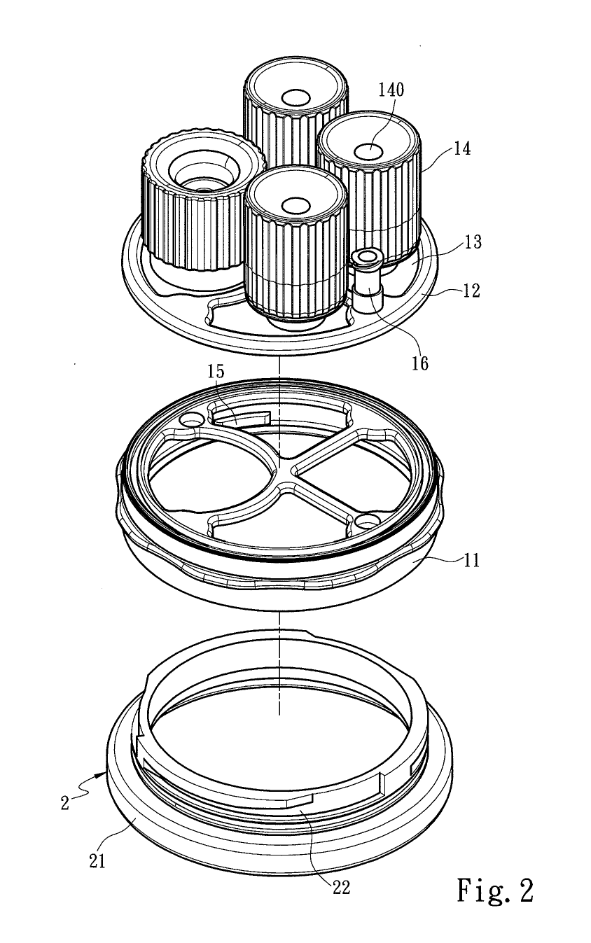

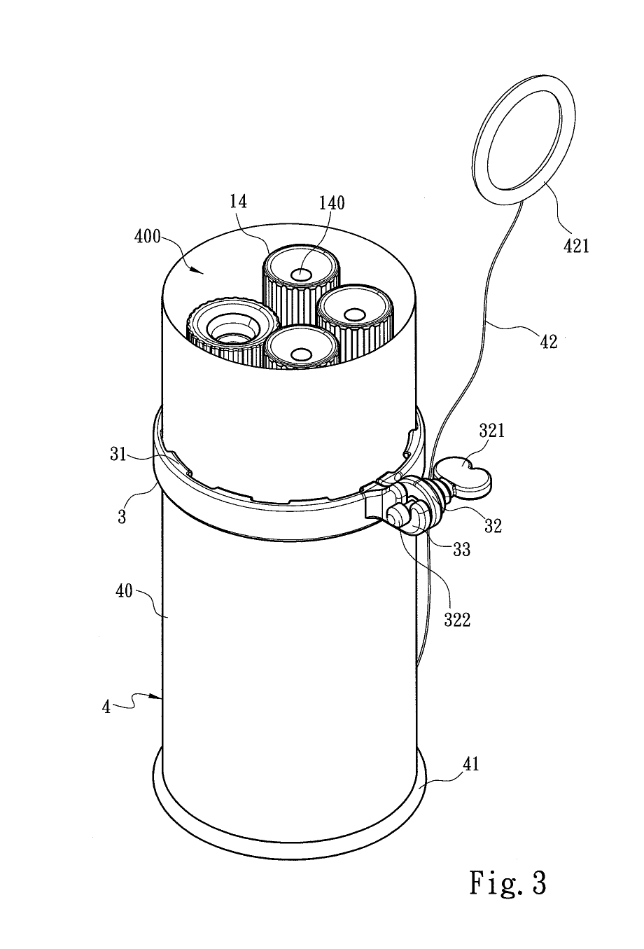

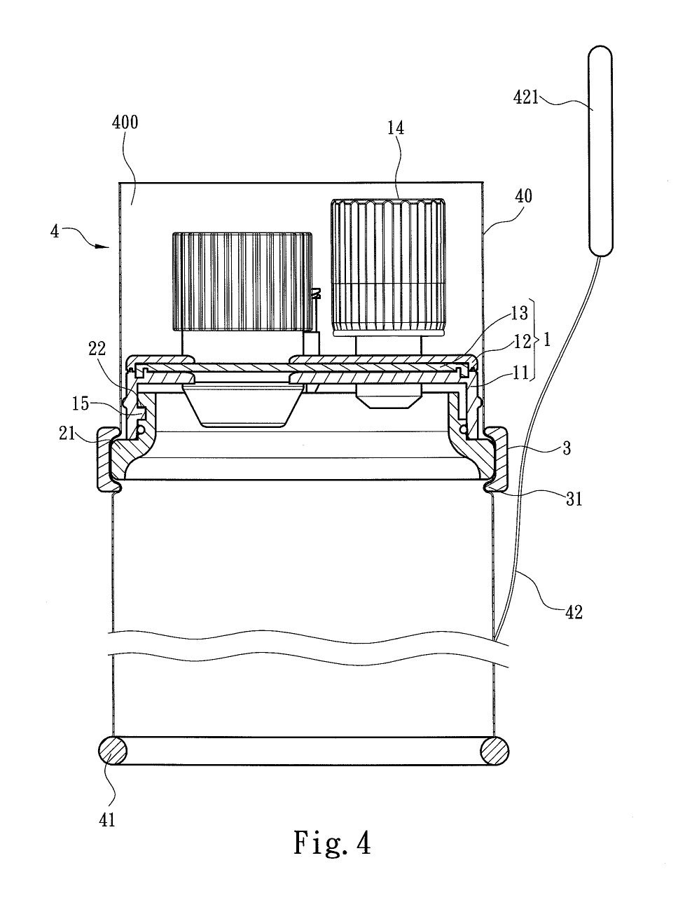

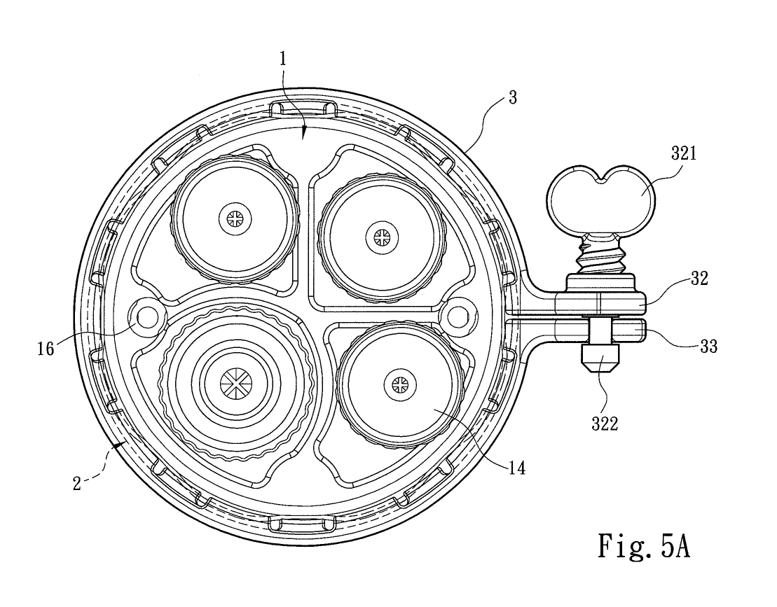

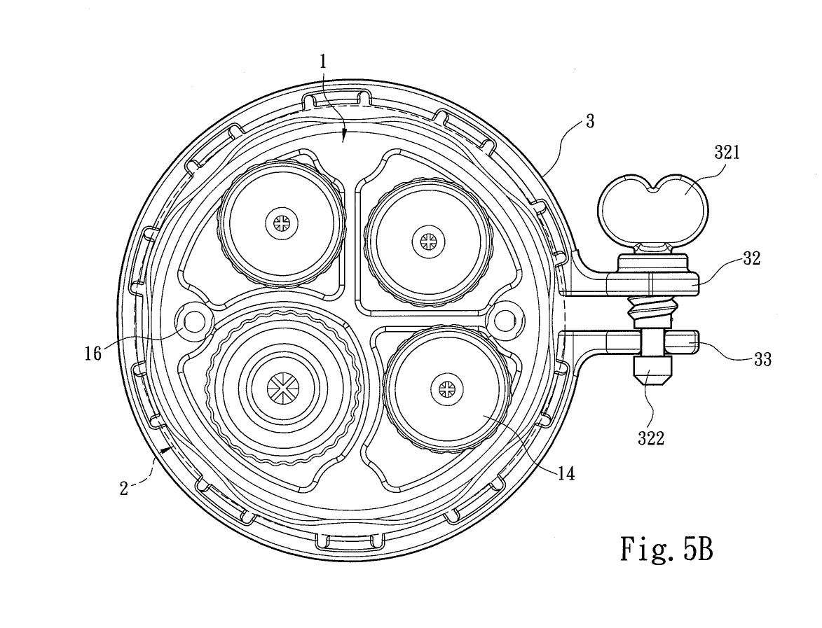

Please refer to FIG. 1, the present invention aims to provide an incision fixation device for single site laparoscopy coupled to a sleeve 4 to be positioned on a surgical incision. The sleeve 4 has an isolation portion 40 and a butting portion 41 at a distal end of the isolation portion 40. During surgical operation, in order to allocate space to facilitate insertion of a surgical instrument (not shown in the drawings) to pass through the surgical incision, the butting portion 41 is inserted through the surgical incision and butted against the inner side of the surgical incision. The inner side of the isolation portion 40 forms a passage 400 passing through the surgical incision. Through the passage 400, surgeons can use the surgical instrument to pass through the surgical incision to perform the surgical operation. In an embodiment of the invention, the butting portion 41 is connected to a drawing rope 42 with a drawing portion 421 connected thereto. When the butting portion 41 is butted against a human body, parts of the drawing rope 42 and drawing portion 421 remain outside the human body, their functions will be discussed later. Also referring to FIGS. 1, 2 and 3, the fixation device includes an upper lid 1, a bracing member 2 and a compact member 3. The bracing member 2 has an anchor end 21 and a first engaging portion 22. When the fixation device is coupled to the sleeve 4, the bracing member 2 is held in the passage 400, and the compact member 3 is coupled on an outer side of the isolation portion 40. The compact member 3 has at least one holding portion 31 on the inner rim, and holds the anchor end 21 with the isolation portion 40 interposed therebetween. The compact member 3 has a first end and a second end that include respectively a first fastening mechanism 32 and a second fastening mechanism 33 coupled with each other to fasten or loosen the compact member 3. Referring to FIGS. 5A and 5B, the second fastening mechanism 33 includes a screwing notch. The first fastening mechanism 32 includes a fastening bolt 321 screwed thereon and rotatably running through the screwing notch. The fastening bolt 321 has a latch end 322 latched on the second fastening mechanism 33. The fastening bolt 321 can be screwed by wrenching to drive the first and second fastening mechanisms 32 and 33 to move against each other to drive the compact member 3 to contract inwards (as shown in FIG. 5A) or expand outwards in reverse operation (as shown in FIG. 5B). Referring to FIGS. 3 and 4, the holding portion 31 of the compact member 3 holds the anchor end 21 with the isolation portion 40 interposed therebetween. Hence, when the compact member 3 is contracted inwards, the holding portion 31 also is contracted corresponding to the anchor end 21 to tightly clamp the isolation portion 40 between the anchor end 21 and the holding portion 31. Therefore, the bracing member 2 and isolation portion 40 are held firmly against each other. On the other hand, when the compact member 3 is expanded outwards, a sliding space is formed between the anchor end 21 and the holding portion 31 for the isolation portion 40, thereby the bracing member 2 can be moved relative to the isolation portion 40. The upper lid 1 has a plurality of guiding portions 14, at least one inflation port 16 and a second engaging portion 15. The inflation port 16 is used to inflate patient's body to provide a room for surgical operation. The second engaging portion 15 is releasably engaged with the first engaging portion 22. FIG. 2 illustrates an embodiment in which the second engaging portion 15 is a flange and the first engaging portion 22 is a track with an one-way opening corresponding to the flange, so that by rotating the upper lid 1, the second engaging portion 15 can be entered into the first engaging portion 22 for engagement. Each guiding portion 14 has an instrument passage 140 to guide a surgical instrument. After the upper lid 1 is coupled with the bracing member 2 through the second engaging portion 15, the instrument passage 140 communicates with the passage 400 to guide the surgical instrument to pass through the surgical incision to perform surgical operation. The instrument passage 140 also can hold a check member to enhance isolation. More specifically, the upper lid 1 can include a base 11, a sealing member 12, an airtight member 13 and the guiding portions 14 mentioned above. The airtight member 13 isolates the passage 400 from the exterior. The guiding portions 14 are located on the airtight member 13. The circumference of the airtight member 13 is clamped and held securely between the coupled base 11 and sealing member 12. The second engaging portion 15 is located on the base 11. The airtight member 13 is preferably made of flexible plastics so that a slight movement space can be provided for the directing portions 14 wedged in the airtight member 13. After surgical operation is finished, the compact member 3 is loosened to separate the upper lid 1, the bracing member 2 and the compact member 3 from the isolation portion 40, and the butting portion 41 can be removed by pulling the drawing rope 42.

By means of the construction set forth above, when in use, the butting portion 41 at the distal end of the sleeve 4 is inserted through the surgical incision and butted against the inner side of a human body. Through loosening the compact member 3, the upper lid 1, the bracing member 2 and the compact member 3 can be slid on the isolation portion 40 to prospective locations desired by medical personnel; then the upper lid 1 and the bracing member 2 can be firmly positioned by contracting the compact member 3 through the first and second fastening mechanisms 32 and 33. In short, by loosening or contracting the compact member 3, the upper lid 1 and the bracing member 2 can be slid on the sleeve 4 to prospective locations to be adjusted as desired, thus resolve the problem of rolling the sleeve 4 in the conventional technique. It also improves the installation of the upper lid 1 and the bracing member 2. Besides, when the resected tissues have to be removed, the upper lid 1 and bracing member 2 can be quickly disassembled and assembled again. As the upper lid 2 includes the guiding portions 14 for providing bracing forces, there is no need to use a trocar to guide the surgical instruments to pass through the surgical incision as the conventional technique does. Without using extra trocar, the workload of medical personnel can be reduced. Moreover, the invention also provides the fine drawing rope 42 on the sleeve 4 to reduce unnecessary harm to the incision of the patient.

While the invention has been described by means of specific embodiments, they are not the limitations of the invention, numerous modifications and variations could be made thereto by those skilled in the art without departing from the scope and spirit of the invention set forth in the claims.

In summation of the above description, the present invention provides a significant improvement over the conventional techniques and complies with the patent application requirements, and is submitted for review and granting of the commensurate patent rights.

What is claimed is:

1. An incision fixation device for single site laparoscopy coupled with a sleeve to be positioned on a surgical incision, the sleeve at least including an isolation portion to form a passage, the fixation device comprising:
a bracing member which is located in the passage and includes an anchor end and a first engaging portion;
a compact member which is coupled on an outer side of the isolation portion and includes at least a holding portion corresponding to the anchor end to be securely mounted onto the isolation portion of the sleeve; and
an upper lid which includes a plurality of guiding portions and a second engaging portion, the second engaging portion being releasably engaged with the first engaging portion, each guiding portion including an instrument passage communicating with the passage to guide a surgical instrument to pass through the surgical incision to perform surgical operation.

2. The incision fixation device of claim 1, wherein the compact member includes a first end and a second end that include respectively a first fastening mechanism and a second fastening mechanism to couple with each other to drive the compact member to fasten or loosen.

3. The incision fixation device of claim 2, wherein the second fastening mechanism includes a screwing notch, the first fastening mechanism including a fastening bolt screwed thereon and passed through the screwing notch to drive the first and second fastening mechanisms to contract inwards.

4. The incision fixation device of claim 1, wherein the upper lid includes an airtight member to isolate the passage from the exterior, the guiding portions being located on the airtight member.

5. The incision fixation device of claim 4, wherein the upper lid further includes a base and a sealing member, the base including the second engaging portion located thereon, the airtight member being located between the base and the sealing member.

6. The incision fixation device of claim 4, wherein the airtight member is made of flexible plastics.

7. The incision fixation device of claim 1, wherein the second engaging portion includes a flange, the first engaging portion including a track with an one-way opening corresponding to the flange, the upper lid being rotated to allow the second engaging portion to engage with the first engaging portion for positioning.

8. The incision fixation device of claim 1, wherein the sleeve includes a butting portion at one end.

9. The incision fixation device of claim 8, wherein the butting portion is connected to a drawing rope which includes a drawing portion.
